(19) United States
(12) Patent Application Publication
(10) Pub. No.: US 2001/0038846 A1
(43) Pub. Date: Nov. 8, 2001

(54) METHOD IF TREATING KELOIDS

(76) Inventor: SANDRA BROWN, SOUTHFIELD, MI (US)

Correspondence Address: THOMAS E ANDERSON, GIFFORD KRASS SPRINKLE, ANDERSON & CITKOWSKI, 280 NORTH OLD WOODWARD SUITE 400, BIRMINGHAM, MI, 48009

(21) Appl. No.: 09/309,033

(22) Filed: May 10, 1999

(51) Int. Cl. A61K 9/00; A61K 6/00; A61K 7/00; A61K 31/19

(52) U.S. Cl. 424/401; 514/557

(57) ABSTRACT

Disclosed is a method for treating keloid scaring. The method includes application of a concentrated solution of glycolic acid in the range of 20-70% by weight to the skin for a period of time sufficient to promote atrophy of the scar. After a sufficient time, the tissue is washed with soap and water to remove the acid and loosen tissue.

This application claims the benefit of U.S. Provisional Application No. 60/085,165, filed May 12, 1998.

### BACKGROUND OF THE INVENTION

1. Field of the Invention

Keloids are raised, hard, generally irregularly shaped scars which form on the skin as a result of trauma including burns, lacerations, abrasions, cryosurgery, vaccinations, and the like. Keloids are characterized by the presence of thick bands of acellular, eosinophilic collagen therein, and are thought to form as a result of a defective healing process. Keloids can occur in people of any race, but are more common in black people.

In some instances, keloids can produce severe physical and psychological symptoms. Keloids are prone to bleeding and itching, and can be extremely sensitive to touch so that contact with clothing, or even water, can produce excruciating pain. Keloids can be highly disfiguring, and their untreatable nature has caused some patients to become recluses or even commit suicide.

In many instances, keloids tend to flatten and cease to itch after several months time, but in other instances, keloids continue to grow sometimes to very large sizes, and can be painful and highly disfiguring.

Prior to the present invention, there has been no completely satisfactory treatment for keloids. Surgery can be used to remove the keloid; but, the new scar which forms after surgery is almost always a keloid and in some instances is even larger. The itching of keloids can, in some instances, be reduced by injecting corticosteroids thereinto; but, such injections only provide temporary relief and are ineffective in reducing keloid size. X-rays have been employed to ablate keloids, but success has been limited.

It will thus be appreciated that keloids are a very significant medical problem which heretofore has not been amenable to reliable medical treatment. The present invention provides a method for the treatment of keloids which is highly effective in reducing their size and severity of their attendant symptoms. In many instances, the treatments of the present invention can completely remove all evidence of keloids and prevent any recurrence. The treatment of the present invention relies upon the use of carboxylic acid materials which are relatively nontoxic and low in cost.

### SUMMARY OF THE INVENTION

The invention is a method for treatment of keloid or burn scars, including the steps of cleansing the skin with soap and water, applying a relatively concentrated solution of glycolic acid to the area of treatment, rewashing the skin to remove the acid treatment and loosen the outer layers of keloid.

### DESCRIPTION OF THE DRAWING

Other objects, features and advantages of the present invention will become apparent by reference to the following detailed description and to the drawings, in which:

FIG. 1 is a flow chart providing a schematic overview of the steps according to the method of the invention.

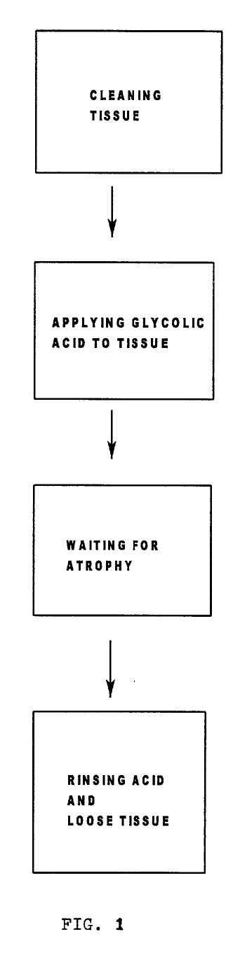

### DETAILED DESCRIPTION OF THE INVENTION

In accord with the present invention, it has been found that keloid or burn scarring may be reduced, and in many instances completely removed, by the application of carboxylic acids thereto. Specifically, it has been found that alphahydroxy acids are highly effective in the practice of the present invention. One particularly preferred acid comprises glycolic acid. While not wishing to be bound by speculation, Applicant presumes that the acids of the present invention are effective insofar as they readily penetrate and remove the thickened layers of skin, in a controlled manner, while promoting and controlling the growth of new tissue layers therebeneath.

In a typical treatment regimen, the affected area of the skin is first cleansed with soap and water, and thereafter contacted with a relatively concentrated solution of glycolic acid. This stage of the treatment is preferably carried out under medical supervision, since excess concentrations of the acid, or excessive contact times, can cause irritation to the keloid which could promote its further growth. Typically, the acid is employed at a concentration in the range of 20 to 70% by weight, with the remainder of the composition comprising distilled water or any other pharmaceutically acceptable carrier such as a lotion base, an alcohol base or the like. While other concentrations may be employed, concentrations below 20% typically are not aggressive enough in action to be used practically at this stage of treatment. Also, concentrations above 70% do tend to produce keloid irritation. The choice of the exact therapeutic concentration will depend upon the size and thickness of the keloid, and the length of contact time the acid is maintained upon the keloid. Most typically, this concentrated acidic treatment of the keloid takes place for a time in the range of 3 to 15 minutes, with 8 to 10 minutes being a most preferred range. The treatment produces a degradation of the outer layers of the keloid manifested as a somewhat frosted appearance to the skin. As treatment progresses, patients often report tingly or prickly feelings in the area, and these sensations may progress to a slight burning feeling. In general, patient discomfort is fairly minimal, and compliance with the treatment regimen is usually not a problem.

After the first stage of treatment is completed, the skin is again washed with a soap and water solution to remove the acid treatment and loosen the outer layers of the keloid.

The patient is then sent home and is generally provided with a conventional hydrocortisone ointment, which is applied to the treated region one or more times per day. The hydrocortisone treatment reduces itching or other discomfort caused by the treatment, or by the underlying keloid. Treatment results in progressive atrophy of the keloid tissue. In some instances, it is desirable to provide the patient with a relatively low concentration of glycolic acid based composition which they apply to the keloid on a daily basis. The weight concentration of this composition is generally in the range of 1 to 10%, and more preferably 2 to 5%.

The aforedescribed treatments are typically repeated on a three to four week basis, and the total duration of the treatment regimen will depend upon the size and thickness of the keloid scar. Typically, treatment is carried out for a period of three months to two years.

Thus, it will be appreciated that the foregoing discussion and examples are illustrative of typical embodiments of the invention, but are not meant to be limitations upon the practice thereof. It is the following claims, including all equivalents, which define the scope of the invention.

I claim:

1. A method for treatment of keloid skin damaged by one of the group of keloid, scar and burn, said method comprising:
applying glycolic acid to said keloid scar, and maintaining said acid in contact with said damaged skin for a period of time sufficient to promote atrophy of at least a portion of said damaged skin.

2. A method as in claim 1, wherein said glycolic acid has a concentration in the range of 20-70% by weight.

3. A method as in claim 1, wherein said glycolic acid has a first concentration in the range of 20-70% by weight, said method including the further steps of washing said acid from said damaged skin, and thereafter applying, to said damaged skin, a second concentration of an acid, which second concentration is lower than said first concentration.

4. A method as in claim 1, including the further step of applying hydrocortisone to said damaged skin.
